SEQUENCE LISTING (13 sequences)

SEQ ID NO: 1 - DNA, 20 bp, Mesocricetus auratus
  misc_binding (1)..(20)
gccccgccccgtcccgcccc

SEQ ID NO: 2 - DNA, 7 bp, Mus musculus
  misc_binding (1)..(7)
cccgccc

SEQ ID NO: 3 - DNA, 7 bp, Mus musculus
  misc_binding (1)..(7)
tccgccc

SEQ ID NO: 4 - DNA, 118 bp, Mesocricetus auratus
  misc_feature (1)..(118)
tccagcaaatgcgttacttcctgccaaaagccagcctccccgcaacccactctcccagaggccccgccccgtcccgccccctcccggcctctcctcgtgctggatcgctccctaagga

SEQ ID NO: 5 - DNA, 220 bp, Mus musculus
  misc_feature (1)..(220)
aggatggacatcgcacatcccctttccacccatatatctttgaggtagggatgcttgtgtttaggcagctcaagaaatctaacccctgactcaggccccacacacacctcgcagaggccccgcctctcagcctgtcccgcccctcgtgctagaccaacccgcacccagaagccccgcccatcgaggacgctccgcccttgttccccccgggattgacgtg

SEQ ID NO: 6 - DNA, 587 bp, Human cytomegalovirus
  enhancer (1)..(587)
ttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattactcgagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 7 - DNA, 204 bp, Human cytomegalovirus
  promoter (1)..(204)
tgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 8 - DNA, 376 bp, Human cytomegalovirus
  enhancer (1)..(376)
tgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctatta

SEQ ID NO: 9 - DNA, 3960 bp, Mesocricetus auratus
  gene (1)..(3960)
ggatccggacaacacccacaccggcccctccaggtccagaaagctggccctgcgagaagcgggactgaaaaggcgtgcgggagccagaaatccaaaagggtgccaaggcatgcgtcctttttccacccagaaataaccccaggctttcaatttgaggttatttcaatatccagcaaatgcgttacttcctgccaaaagccagcctccccgcaacccactctcccagaggccccgccccgtcccgccccctcccggcctctcctcgtgctggatcgctccctaaggacgccccgctccagaagccccacctaccaaggacgccccacccttgttcccggactggtatgaccccagcctgctgacatccctccgccctttctcgtgcacgcggctatggcggaatctgagttgcagctggtggcgcagcgatccgcagtttccccgacttccccatccccggcgtgctgtttaggtgagatcacgagccagcaaggcgttggagccctgttcctgggctcccggcgaggcgcatgggcagtctcggggatcttgtggggtctccgcccccctttccccggccaccagcctctccttgttcccagggatatctcgcccctcctgaaggaccccgcctccttccgagcttccatccgcctcctggccagtcaccttaagtccacgcatggcggcaagatcgactacatcgcaggcgagtggccatgccaggccgtgctggtcccccactgtgcaggctcccctcccttcccttatgtcaccctcagtccatcccacacccatcccttctttcttcaacccctaatcctcttttagcactctgtttctcttgccttggtccctcgttgaccctggatgagtactgcgtctcccacccctgctaggtctctgacctccgccctcagtgcctgttctactagagatgaactctgctcggtccttgttcagggccagcccttctctcttggggtgtgaagttggctagcagcctggaggcaggactgtaagaagcatacgtgtgctttgagaactttgaggaaggccctggaacctccttgctaggagtagcacctaagatgaactagatgctaaaaaatgctgtatctttggggcacacgagggcatgcctgggcaggcttagagcctggtagtctcaggggctgcaccaaagtgtaattcttgtgctaaataactttcacttaccagtgccaagcacgggcttcagaaacaccctagggtcgctgaatgtccaccaggggagtcagacatgtccagagggtgagaaccccagagaattcggtagccctgacatgtgctacaattactgatgcccacttcctactggttcctcctggccatacctcaggaattagggcatgctttctgcctgctacagtagctcatcctccctggaagtgaccccagacatataccctgaactgtaaccgataaagtgcgcctgggcagatgtatttgagaggtggcaaaagtaaaccataggtgtccccgagctagatacagaaggcagataacatccccaaggctaagctgctgccccaatagccatcagccttctagttatagctagtaagacctagtattcctggtcaatactattcactcaatccttacacctcagccctaacacgccccctctctcatcctaacaggcctagactccaggggattcttgtttggcccctccctagctcaggagctgggcctgggctgtgtgctcatccggaagcgagggaagctgccaggccccacagtgtcagcctcctatgctctcgagtatggcaaggtaagcaggcagtgggtagctgtctaggagtaaatgtgggggctcagagaggttaagtcatcaggccaggtttataccaccaggaaacatggagaagctaggggtggtggtcacttgttagctactagactctcactctacttcctgtctgcaggctgaactagaaatccagaaagacgccttagaacctggccagaaagtggttgttgtagatgatctcctggccactggaggtaagagccactctgtagcataaagaggtttcaaagggataagccctatccggggtgctgactaagcaagagccttactacctgtgtctttcctcgtcccttcaccccaggaaccatgtgcgctgcctgtgagctgctgggccagctacaggctgaggtggtggagtgtgtgagcctggtggagctgacctcacttaagggcagagagaagctaggatcagtaccattcttctctctcctgcaatatgagtgactggaatggttgctgcatccctgccctcagcatctacggttgaacagtggctcagccttaccaaagtgacctttgtgagccaccagctgcactttctctaactctattcactcatttctttggtcagctgatggccatgcctatggaccacctgggtccttgcattttatatatgtgctgagtcctggagcagagcagagctactgtgggttatgacacagcagatcaataaatagtttggtacatatggtgcttcctgttgtcttgttgcatggatcacgttccatagctgccaggtgacagtccctgtcccttgggattccctgtgcaaatgttctccacagagtcaatcccacacactgccccggccgtagggtcttatagaaccagaatccgttttaagcaaaaggctttcatcagtagtctctagagtgacatgatgggaacaggggctcagtgtcaccttgcagacctcttcaaaacaggtggaaaccagtaggcacagctctgtatagatttcacagcctctaggaagttttctgggccattcctttaggaccaaaatggagagagtagcccaagggaataccttattcatattttgttgaacatcaataccaggttgtttgtagggatgctgagccacacccccagcaagactgttcccatccagacttttactggcctggtcttgctagccatagaagaaaacattgcaacaacagcagggcatcatttataggtattttatggtgacccattcagtttaatttctgtattacttttatttaaatcttacgtgactttattaaaagccaagattaacagtcacatccaaggtacagaaggttaaataaaaattttgatctcaaaacctatagttatttccattatatacagaaggcccaatttcactcttgccattcagaactctctgtccttatgaaagctggccagaagcccaagttctctgtgtgggtcttaaggctctaagcaagacagcagctttcatgagcatggagaacaggctaggctttggcctaaggctcccaactggaccttgccagtacctagatgagtagtttgtgggacgggcatctaacatactctgctctccaagtctacttccccaacacccacacaagtgctgggtctggggtacccaaagcctcaggaaccaatttagcactttatgggcacaattgctgcccatactggcagtaagcccttgggcctgtgacactgcgagggatgctcaagtgcattgtactgataaagggaacagactatcaggtaaagaaatgagtcacaacccctcagtgtggatctggtgggtcagcctttcagtgaggccagctaagtcagcagctttgtccagcttgatgtaggtgtccatgcgaatccgatgcaggctcagccagtctggcagcaactcagataggagcaacacatgtttctccatctcccctgtggaagtgcattgtggttaactgaagcacagtgcctggatgacttgcttaactcacttacccagcctggtgccttacacccctcaaggcttaacctgtgtgcagtggatccaagcagagcctgggccctgctctcaaacaccagggggatgaacagcaagggcaagacacatatacgccaagctt

SEQ ID NO: 10 - DNA, 3066 bp, Mus musculus
  gene (1)..(3066)
gaattcatgctcacgggctcacaggaaggtccaagaaggaatgtttagaatccattggaccctccccacaccctctcctttgatggagcatgggccaatttggaggatatcttttgagtaattgcaactgcactgaagatgataatggccattatactcagaggacagtctttccacaccactacctatagacccaagtactgtgctgggaaggtagaaccccagttctgtctctggctatcaggaccttctggttccaccccaaaacgaggagggcacattctgttgcaatgcacaggagtgtctgtggtctcagagaaggcattccttacccgccctgctaccctgctttcccctgcgctctagcccacacacagtgcactcccacctctggacctagactatccatcagctcccttccggtaatttcaggaaagcaggggctgaatctcaggcccttgtactatgcgcgagggaaggaacgcaaggccaaaccactccagcggacctgggcaagacccgtccctgctcccccaggtccagaagactagcccctggaaaagcaggactgaaaaagcgtgtgtggggcaaaaccaaaaaaggatggacatcgcacatcccctttccacccatatatctttgaggtagggatgcttgtgtttaggcagctcaagaaatctaacccctgactcaggccccacacacacctcgcagaggccccgcctctcagcctgtcccgcccctcgtgctagaccaacccgcacccagaagccccgcccatcgaggacgctccgcccttgttccccccgggattgacgtgagtttagcgtgctgatacctacctcctccctgcctcctacacgcacgcggccatgtcggaacctgagttgaaactggtggcgcggcgcatccgcgtcttccccgacttcccaatcccgggcgtgctgttcaggtgcggtcacgagccggcgaggcgttggcgctgtacgctcatcccccggcgcaggcggtaggcagcctcggggatcttgcggggcctctgcccggccacacgcgggtcactctcctgtccttgttcctagggatatctcgcccctcttgaaagacccggactccttccgagcttccatccgcctcttggccagtcacctgaagtccacgcacagcggcaagatcgactacatcgcaggcgagtggccttgctaggtcgtgctcgtcccccacggtcctagcccctatcccctttccccctcgtgtcacccacagtctgccccacacccatccattcttcttcgacctctgacacttcctccttggttcctcactgccttggacgcttgttcaccctggatgaactatgtaggagtctcccttccctgctaggtaccctaaggcatctgccctcggtgcttgttcctagagacgaactctgctctgtccttgtgtccagaaccaggcctccctcttttagggcacaaagctggccagcatcctgacagcaggctgggagaccctggaacctccagatgacggacatccttgcttaggggtagcctctgggatgaactagatactaaaaattaggtaaccttggttgggcgtggcgtgcctgggcagacctcaagcctggtagcttcaggggctgtttctccccaggactacaccggggcatctttctcttgttccctcacacaagcttgtgttaaacaactgctgtctacttggctccatgcctgagcttgagaaacaccctaggacagctgaatgtccaccaggagtgtccagagggagggtgggcaccccagagaacagagtggccttggtaagtgctcggggaccacagactttgccacttcacttcctattggtacccttggccatgctccagaaattagggcatgtatgtatccttcccacgacagctagatgctgcatttgaaggtggcaagaccaccataggtggccctgagctgttcagaaggcaggtaggatccccaaggctgagatgatgagttgatggctacccagtagccatcaacgttcttctaaccgtagtcagcaagacctagtgttcctagcaagtgttgacctcgcccatacttggcctctagattcccatgcccctcagctccatcccacaaccttccctccttaccctaacaggtctagactccaggggcttcctgtttggcccttccctagctcaggagctgggcgtgggctgtgtgctcatccggaaacaggggaagctgccgggccccactgtgtcagcctcctattctctggagtatgggaaggtaagcgagctgtgtgtagaggaagggcagggtcttatcacggctaccagtgtctaggagtaaatgtgggtgctcagagaggttgagacattgggtcaggtttacaccacccagaaacgctcgagcctagggaggtggccacttgttcgcgcctagactctgtcttacactacttcctgtctgcaggctgagctggaaatccagaaagatgccttggaacccgggcagagagtggtcattgtggatgacctcctggccacaggaggtaaagaaccaacccaagacaaacagacttcaaagggccagaccctgtcctgggtgctgactaagcaaagagcttgaacacctcctctttctctgtcccttccccccaggaaccatgtttgcggcctgtgacctgctgcaccagctccgggctgaagtggtggagtgtgtgagcctggtggagctgacctcgctgaagggcagggagaggctaggacctataccattcttctctctcctccagtatgactgaggagctggctagatggtcacacccctgctcccagcagcactaggaactgcttggtggctcagcctaggcgcctaagtgacctttgtgagctaccggccgcccttttgtgagtgttatcactcattcctttggtcagctgatccgccgtgcctgtggacccctggatccttgtactttgtacacgtcccacacaccctggagcatagcagagctgtgctactggagatcaataaaccgttttgatatgcatgc

SEQ ID NO: 11 - DNA, 543 bp, Mus musculus
  exon (1)..(543)
atgtcggaacctgagttgaaactggtggcgcggcgcatccgcgtcttccccgacttcccaatcccgggcgtgctgttcagggatatctcgcccctcttgaaagacccggactccttccgagcttccatccgcctcttggccagtcacctgaagtccacgcacagcggcaagatcgactacatcgcaggtctagactccaggggcttcctgtttggcccttccctagctcaggagctgggcgtgggctgtgtgctcatccggaaacaggggaagctgccgggccccactgtgtcagcctcctattctctggagtatgggaaggctgagctggaaatccagaaagatgccttggaacccgggcagagagtggtcattgtggatgacctcctggccacaggaggaaccatgtttgcggcctgtgacctgctgcaccagctccgggctgaagtggtggagtgtgtgagcctggtggagctgacctcgctgaagggcagggagaggctaggacctataccattcttctctctcctccagtatgactga

SEQ ID NO: 12 - DNA, 70 bp, Artificial Sequence
  Synthesized
tccagcaaatgcgttacttcctgccaaaagccagcctccccgcaacccactctcccagaggccccgcccc

SEQ ID NO: 13 - DNA, 68 bp, Artificial Sequence
  Synthesized
tccttagggagcgatccagcacgaggagaggccgggagggggcgggacggggcggggcctctgggaga

(19) United States
(12) Patent Application Publication
(10) Pub. No.: US 2014/0017726 A1
(43) Pub. Date: Jan. 16, 2014

(54) MODIFIED HUMAN CMV PROMOTERS THAT ARE RESISTANT TO GENE SILENCING

(71) Applicants: Yuansheng Yang, Centros (SG); Mariati Mariati, Centros (SG)

(72) Inventors: Yuansheng Yang, Centros (SG); Mariati Mariati, Centros (SG)

(73) Assignee: AGENCY FOR SCIENCE, TECHNOLOGY AND RESEARCH, Singapore (SG)

(21) Appl. No.: 13/996,345

(22) PCT Filed: Dec. 22, 2011
(86) PCT No.: PCT/SG2011/000449

§ 371 Date: Sep. 19, 2013

Related U.S. Application Data
(60) Provisional application No. 61/427,121, filed on Dec. 24, 2010.

(51) Int. Cl. C12N 15/86

(52) U.S. Cl. CPC C12N 15/86
USPC 435/69.1; 435/320.1

(57) ABSTRACT

The invention relates to a nucleic acid molecule comprising a functional promoter of a herpesvirus, a functional enhancer of a herpesvirus, and one or more internal elements of the CpG island of the aprt (adenine phosphoribosyl transferase) gene and/or a functional variant thereof. A method of producing a desired polypeptide using the nucleic acid molecule, a vector and a host cell containing the nucleic acid molecule are also disclosed.

### CROSS-REFERENCE TO RELATED APPLICATION

This application claims the benefit of priority of U.S. provisional application No. 61/427,121 filed Dec. 24, 2010, the contents of it being hereby incorporated by reference in its entirety for all purposes.

### FIELD OF THE INVENTION

The present invention lies in the field of molecular biology, virology and gene therapy, and particularly relates to a nucleic acid molecule containing a functional promoter of a herpesvirus, a functional enhancer of a herpesvirus and one or more internal elements of the CpG island of the aprt (adenine phosphoribosyl transferase) gene and/or a functional variant thereof, including uses of the nucleic acid molecule and methods of producing a polypeptide or protein of interest.

### BACKGROUND OF THE INVENTION

The majority of studies in gene therapy research to date have utilized viral promoters. In general, strong viral promoters are required for efficient viral propagation, and they frequently induce much higher levels of transcription than eukaryotic promoters by using mechanisms to control and recruit host transcription machinery. Moreover, viral promoters tend to be far more compact and hence easier to manipulate and accommodate into gene therapy vectors.

Human cytomegalovirus major immediate early gene promoter (hCMV) has been widely used in both academic research and industrial applications for high level recombinant protein production in mammalian cells. For instance, the enhancer/promoter element driving immediate/early gene expression in hCMV has been widely used in biotechnology for driving expression of heterologous genes in eukaryotic expression vectors. Other viral promoters in use include the simian virus 40 (SV40), Rous sarcoma virus long terminal repeat (RSV-LTR), Moloney murine leukaemia virus (MoMLV) LTR, and other retroviral LTR promoters. The use of viral promoters has thus been widespread and successful in vitro and for certain applications in vivo. However, eukaryotic cells have evolved mechanisms to detect and silence viral transgene expression. Viral promoters have manifested a frequent inability to sustain transgene expression in vivo, but despite considerable evidence that viral promoters are prone to inactivation and silencing in vitro and in vivo, a large proportion of gene therapy applications continue to utilize them to drive transgene expression. Nonetheless, it is a general problem in that heterologous genes upon transduction into mammalian cells are expressed to a sufficient level transiently which is then suppressed and gradually silenced in stably transfected cell lines during long term culture. Gene silencing is therefore a major obstacle in gene therapy.

The loss in transgene expression is largely attributable to transcriptional silencing which involves methylation at CpG DNA sequences, histone deacetylation or chromatin condensation in the vicinity of the integration site. CpG islands are usually at least 200 bp long, GC rich, and contain at least about 60% frequency of CpG dinucleotides. DNA methylation is a covalent modification in which the 5′ position of cytosine is methylated. In mammals, this modification occurs at CpG dinucleotides. In open chromatin where histones are widely dispersed, DNA is unmethylated, represented by open circles (FIG. 1), gene is actively expressed. Upon methylation, methyl binding (MBD) proteins recruit histone modification proteins and co-repressors to the methylated region, causing chromatin to become condensed, and the gene is silenced.

Various anti-methylation techniques have been applied to prevent gene silencing. For example, Senigl, H and colleagues (Senigl, H; Plachy, J.; Hejnar, J., The core element of a CpG island protects avian sarcoma and leukosis virus-derived vectors from transcriptional silencing (2008) Journal of Virology, 82: 7818-7827) showed that insertion of the internal element (IE) of a CpG island between the enhancer and promoter of a small sized viral promoter, RSV LTR (˜200 bases) prevented gene silencing. However, this strategy may not work for larger size promoters since it has been reported that IE can protect only about 150 bases from methylation (Siegfried, Z.; Eden, S., etc. DNA methylation represses transcription in vivo (1998) Nature genetics 22: 203-206).

UCOE (Ubiquitous Chromatin Opening Element) has been used in research and biotechnological applications such as recombinant protein products in cell culture. UCOE confer an increased proportion of productive gene integration events with improvements in the level and stability of transgene expression. Although UCOE was referred in the art as CpG island related elements (WO 2006/095156), UCOE is known to have a different function that is to structurally loosen chromatin and thus enable expression even if genes are integrated into heterochromatin. It is thus not clear whether UCOE can prevent DNA methylation of the respective nucleic acid.

Generation of cell lines that can maintain high productivity during the extended scale-up period is of the utmost importance for biopharmaceutical manufacturing. Production instability can affect product yield and product consistency, compromising regulatory approval of the product. Most clones generated using currently available expression vectors have unstable productivity. As a result, a large number of clones need to be screened to obtain stable high-producing cell lines. It has been indicated in previous literature reports that the loss in productivity was mainly due to transcriptional silencing which involved methylation of CpG dinucleotides within promoters.

Given the importance of recombinant protein expression in biotechnology, there remains a need for improved expression vectors comprising improved promoter or enhancer combinations.

### SUMMARY OF THE INVENTION

In one aspect, the invention provides a nucleic acid molecule comprising a functional promoter of a herpesvirus, a functional enhancer of a herpesvirus, and one or more internal elements of the CpG island of the aprt (adenine phosphoribosyl transferase) gene and/or a functional variant thereof.

In a second aspect, the invention provides a method of producing a desired polypeptide. The method includes providing a nucleic acid molecule as defined herein, wherein the nucleic acid molecule further comprises a nucleotide sequence encoding the desired polypeptide, the nucleotide sequence being operably linked to the promoter of the herpesvirus and the enhancer of the herpesvirus, and allowing expression of the desired polypeptide.

In a third aspect, the invention provides a vector comprising the nucleic acid molecule as defined herein.

In a fourth aspect, the invention provides a host cell comprising the nucleic acid molecule as defined herein.

In a fifth aspect, the invention provides use of the nucleic acid molecule as defined herein for enhancing the expression of a polypeptide of interest, wherein the nucleic acid molecule comprises a nucleotide sequence coding for the polypeptide of interest, the nucleotide sequence being operably linked to the promoter of the herpesvirus and the enhancer of the herpesvirus.

### BRIEF DESCRIPTION OF THE DRAWINGS

The invention will be better understood with reference to the detailed description when considered in conjunction with the non-limiting examples and the accompanying drawings, in which:

FIG. 1 illustrates a schematic representation of the mechanism behind transcriptional gene silencing. FIG. 1A shows the covalent modification of a nucleotide in which the 5′ position of cytosine is methylated. FIG. 1B shows the DNA methylation occurring at CpG dinucleotides. In open chromatin where histones (“H3”) are widely dispersed, DNA is unmethylated, represented by open circles, gene is actively expressed. Upon methylation, methyl binding (“MBD”) proteins recruit histone modification proteins (such as “HDAC”: histone deacetylases) and co-repressors (such as “HP1”: heterochromatin protein 1) to the methylated region, causing chromatin to become condensed, and gene is silenced.

FIG. 2 shows the nucleotide sequence (SEQ ID NO: 4) of the internal element (IE) of the CpG island found on hamster APRT gene. The internal element (IE) is a small region of CpG island of hamster APRT gene (Brandeis, M.; Frank, D.; Keshet, I., etc. Sp1 Elements Protect A Cpg Island From De-Novo Methylation (1994) Nature 371: 435-438).

FIG. 3A shows the nucleotide sequence (SEQ ID NO: 6) containing human cytomegalovirus (CMV) enhancer and promoter. The CG dinucleotides are indicated in bold, which all could be methylated resulting in gene silencing. The CTCGAG sequence that is underlined and bold indicates the XhoI site generated using site-directed mutagenesis between the enhancer and promoter for insertion of IE.

FIG. 3B shows a schematic representation of the nucleotide sequence of FIG. 3A. Each circle refers to a CG dinucleotide in the sequence. The CG dinucleotides all could be methylated resulting in gene silencing.

FIG. 4 shows a schematic representation of twelve different nucleic acid molecules according to various embodiments of the invention, in which the one or two IEs of the CpG island of the aprt gene is/are inserted upstream of enhancer, between the enhancer and promoter (P), and downstream of promoter (P) in forward or reverse orientations, in order to study the location, orientation, and copy number effects on IE's ability of preventing gene silencing and whether insertion of IE affect promoter strength. The wild type CMV is referred as WT CMV and modified CMV is referred as IE CMV later on.

FIG. 5 shows the vector sequences containing the CMV enhancer and promoter (P) regions for expression of cloned DNA inserts in mammalian cells. MluI is for insertion of IE upstream of CMV enhancer, XhoI is for insertion of IE between CMV enhancer and promoter, NheI is for insertion of IE downstream of promoter. IRESatt, attenuated internal ribosome entry site (IRES). IRES allows expression of multiple genes under the control of one promoter. Attenuation of IRES's translation efficiency is for more efficient selection of high producing clones. mNPT, mutated neomycin with weakened enzyme activity which allows more efficient selection of high producing clones.

FIG. 6 shows a schematic process of clone generation and evaluating the long term expression of the nucleic acid molecules of the invention in cell culture.

FIG. 7 shows the comparison of strength of the twelve nucleic acid molecules containing the modified CMV promoter (“IE CMV”, see FIG. 4) with the wild type CMV promoter (“WT CMV”) in transient (FIG. 7A) and stable gene expression (FIG. 7B). The objective of insertion of IE into CMV was to enhance expression stability but without compromising expression level. The strength of the modified CMV promoters in expressing a gene was compared with the WT CMV in transient and stable transfections using GFP as a reporter gene. The mean fluorescence intensity of transiently transfected pool and stable expressing clones was measured using FACS. Significant differences relative to the WT CMV at a 95% confidence level are denoted by asterisks. Without wishing to be bound by theory, insertion of IE downstream of CMV decreased expression level in transient transfection, while insertion into other locations appear to have minor effect. In stable transfections, UF2, UR2, and MR1 had comparable expression level with wild type CMV.

FIG. 8 shows the percentage of GFP positive cells at week 8 and 0 of a representative clone B4 generated using WT CMV. At week 0, all cells expressed GFP. After 8 weeks passaging without selection reagent G418, only 34% cells still expressed GFP.

FIG. 9 shows the percentage of GFP positive cells at week 0 and 8 of a representative clone B8 using a IE CMV promoter (MF2) according to various embodiments of the invention. The IE CMV promoter (MF2) contains 2 IEs inserted between the enhancer and mini-promoter (FIG. 9A). At week 0, all cells expressed GFP. After 8 weeks passaging without selection reagent G418, all cells still expressed GFP.

FIG. 10 shows the histograms of a representative clone B4 generated using WT CMV at week 0 and week 8. The retention of GFP expression (%) is calculated as the ratio of mean fluorescence intensity (MFI) at week 8 over that at week 0 as follows:

$$Retention of GFP expression \% = \frac{MFI ( week 8 )}{MFI ( week 0 )}$$

FIG. 11 shows the comparison of maintaining GFP expression using the nucleic acid molecules containing the modified CMV promoters (IE CMV, see FIG. 4) and the wild type CMV (WT CMV) during 8 week-passage in CHO cells using GFP as reporter gene. 9 clones were isolated from 3 stable pools for each vector. They were cultured over 8 weeks without the presence of selection pressure. The GFP expression of each clone generated using the WT and IE CMV promoters was monitored over 8 weeks by using FACS as a measure of production stability. FIG. 11A refers to the percentage of GFP positive cells. FIG. 11B refers to percentage retention of GFP expression. Before stability testing, all clones had 100% of GFP positive cells. After 8-week passaging, GFP positive cells in most wild type clones dropped below 100%, and on average, less than 20% of their original expression levels were maintained after 8 weeks passaging. On the contrary, most clones generated using the IE CMV, such as UF2, UR2, MF2, MR1, MR2, and DR2 still had close to 100% GFP positive cells, and on average, close to or over 40% of their original expression levels were maintained in clones generated using UF1, UR2, MR1, MR2, DF2, and DR2.

FIG. 12 shows the schematic representation of three nucleic acid molecules containing the modified hCMV promoters (IE CMV) according to various embodiments of the invention namely, “UR2” wherein 2 IEs were inserted upstream of the enhancer in reverse orientation; “MR1” wherein 1 IE was inserted between the enhancer and promoter in reverse orientation; and “UR2+MR1” containing 2 IEs (reverse orientations) inserted upstream of enhancer and 1 IE (reverse orientation) inserted between the enhancer and promoter. 18 clones were isolated from 3 stably transfected pool for each vector for comparison of strength and maintenance of expression level during long term culture.

FIG. 13 shows the comparison of the strength of the modified CMVs (IE CMVs) namely MR1, UR2 and UR2+MR1 (see FIG. 12) with the wild type (WT CMV) in stable expression. Consistent with results in FIG. 11, UR2 and MR1 showed comparable strength to WT CMV in expressing a gene. However, combination of UR2+MR1 resulted in lower gene expression levels.

FIG. 14 shows the comparison of the modified CMVs namely MR1, UR2 and UR2+MR1 with the wild type (WT CMV) for maintaining proportion of GFP positive cells after 8 week-passage. Consistent with results in FIG. 11, UR2 and MR1 enhanced proportion of GFP positive cells compared to WT CMV during 8 week-passaging. However, combination of UR2+MR1 did not further enhance expression stability.

FIG. 15 shows the comparison of the modified CMVs namely MR1, UR2 and UR2+MR1 with the wild type (WT CMV) for retention of expression after 8 week-passage. Consistent with results in FIG. 11, UR2 and MR1 enhanced expression stability compared to WT CMV during 8 week-passaging. However, combination of UR2+MR1 did not further enhance expression stability.

FIG. 16 shows a schematic representation of two vector sequences as follows: A) “wild type mCE’ containing mouse CMV enhancer and human EF1α promoter (mcE ordered from Invivogen); and B) “mCE+2IE” in which 2 IEs were applied on the promoter consisting of the mouse CMV enhancer and human EF1α promoter. 9 clones were isolated from 3 stably transfected pool for each vector for comparison of strength and maintenance of expression level during long term culture.

FIG. 17 shows the comparison of strength of the modified mCE (mCE+2IE) with wild type mCE in stable expression. Insertion of 2IE into mCE resulted in lower expression level as compared to the wild type mCE.

FIG. 18 shows the comparison of the modified mCE (mCE+2IE) with wild type mCE for maintaining proportion of GFP positive cells after 8 week-passage. Insertion of 2IE into mCE resulted in lower proportion of GFP positive cells as compared to the wild type mCE.

FIG. 19 shows the comparison of the modified mCE (mCE+2IE) with wild type mCE for retention of expression after 8 week-passage. It appears that the insertion of 2 IE between the enhancer and promoter did not improve GFP expression stability. These results, together with those in FIG. 18, indicated that the effect of the IE is promoter specific.

FIG. 20 illustrates the anti-silencing mechanisms of the nucleic acid molecule MR1 containing 1 IE in the reverse orientation inserted between the enhancer and mini-promoter (see FIG. 4). Three clones generated using the WT CMV and MR1 respectively were characterized for changes in GFP expression level, gene copy number, and mRNA level during 8 weeks passaging. The GFP gene copy number and mRNA level were determined using qRT-PCR and normalized to β-actin. FIG. 20A shows the changes in GFP expression levels, FIG. 20B shows the changes in GFP gene copy numbers and FIG. 20C shows the changes in GFP mRNA levels between WT CMV and MR1. GFP gene copy numbers in clones generated using WT CMV remained unchanged during long term culture while mRNA levels dropped and correlated to the decrease in mean fluorescence intensity, suggesting the loss in GFP expression level was due to transcriptional silencing. In contrast, clones generated using MR1 maintained GFP gene copy number and mRNA levels during long term culture, thus exhibited less drop in expression levels.

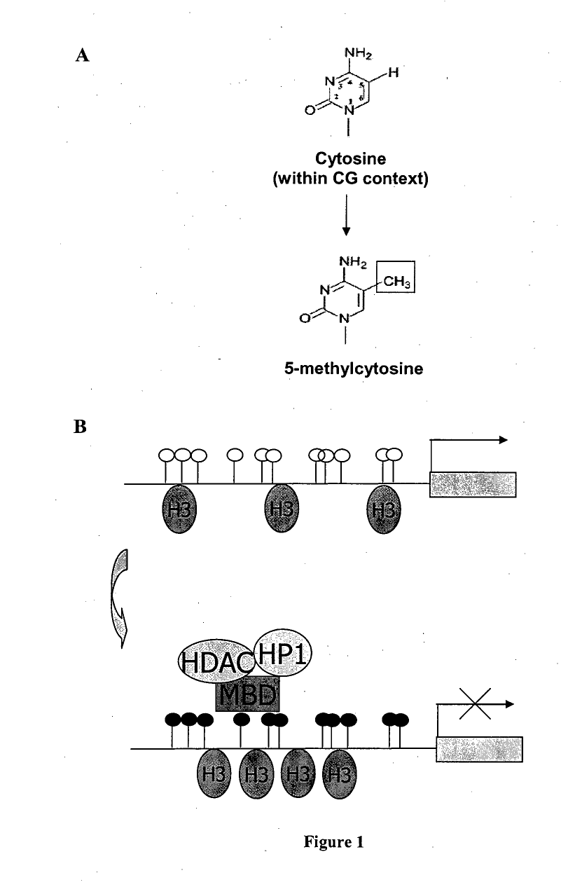

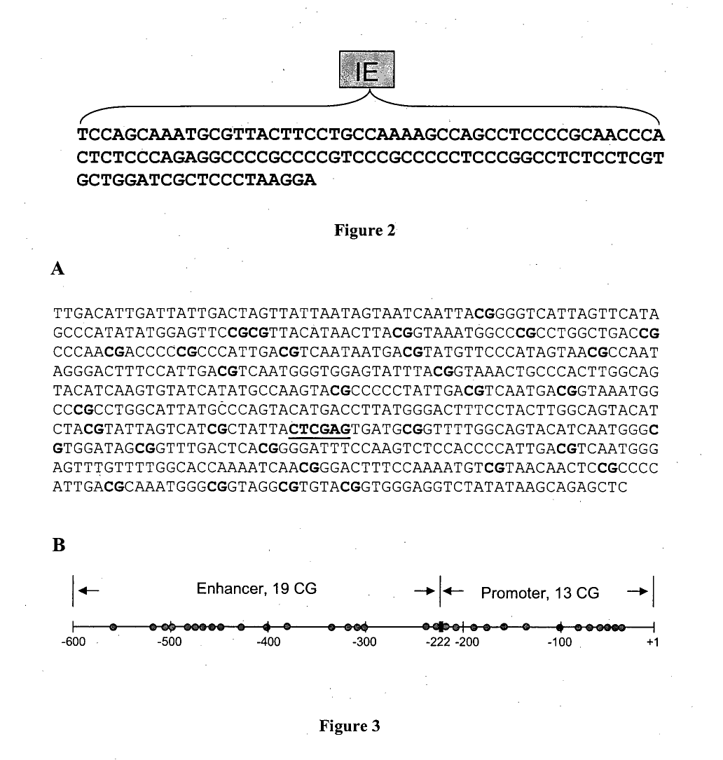

### DETAILED DESCRIPTION OF THE INVENTION

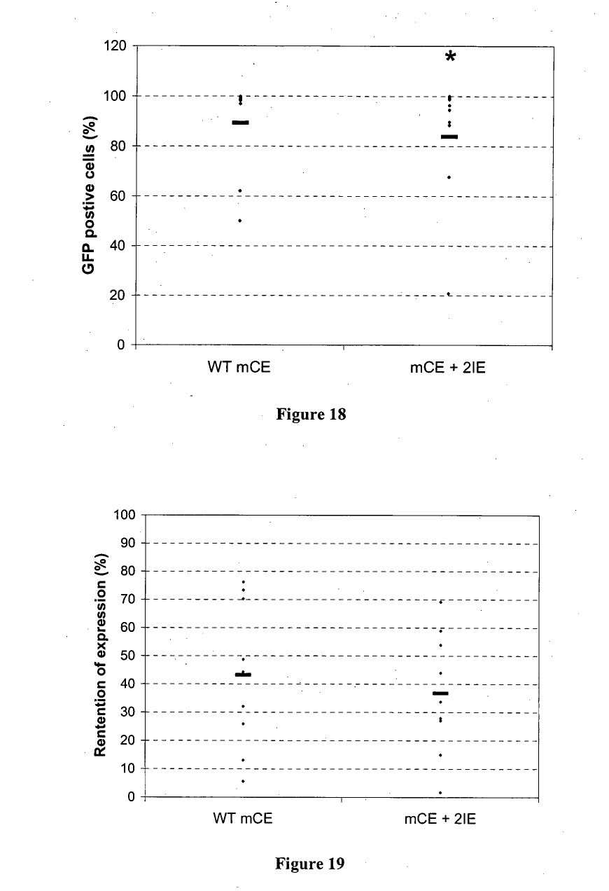

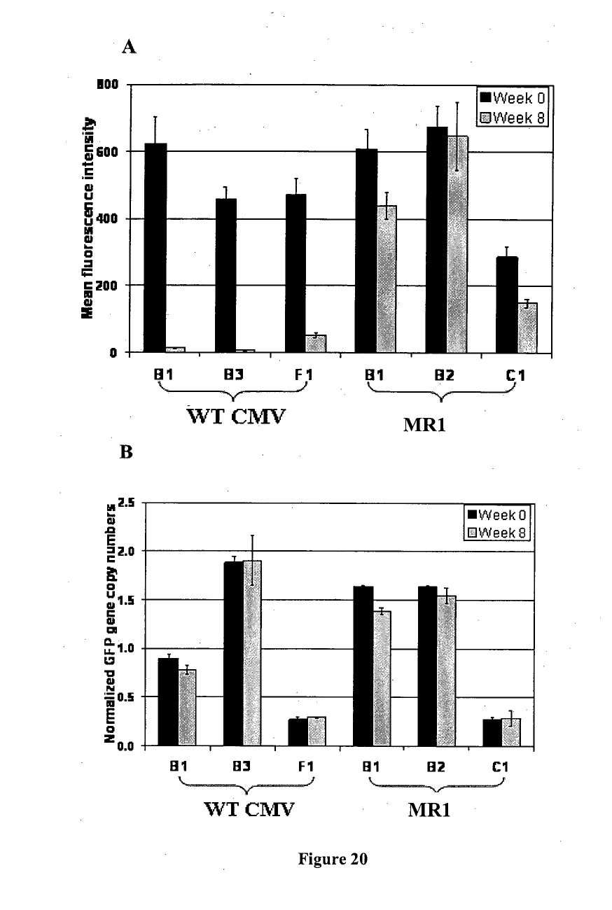

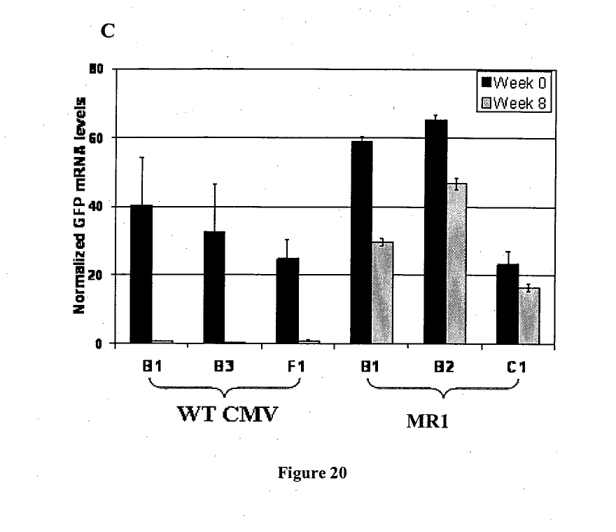

The invention is based on the surprising finding that the insertion of one or more core CpG island elements (IE) into the promoter region of a herpesvirus at specified locations and orientations can prevent a nucleic acid molecule, in particular, DNA, from methylation, so that gene expression levels can be maintained but without compromising the promoter strength. In particular, the modified promoter region of the herpesvirus is more resistant to gene silencing due to the presence of one or more IEs. Therefore, this strategy can be applicable for protection of large sized promoters. However, it was also found that not all so-called CpG island related elements are able to perform the function of preventing gene silencing in any nucleic acid sequence (see for example, FIGS. 17-19), but that this functionality may be promoter-specific. It was thus a surprising finding that an internal element of the aprt (adenine phosphoribosyl transferase) gene can prevent gene silencing within the nucleic acid sequence of a herpesvirus. In this context, the SP1 binding sites within the internal element of the mouse aprt gene were found to be critical for demethylation.

The present invention provides a nucleic acid molecule comprising a functional promoter of a herpesvirus, a functional enhancer of a herpesvirus, and one or more internal elements (IE) of the CpG island of the aprt (adenine phosphoribosyl transferase) gene and/or a functional variant thereof.

The aprt gene may be derived from any organism or species and can for example be selected from the group consisting of the hamster aprt gene, the mouse aprt gene, the rat aprt gene, the human aprt gene, the bovine aprt gene, the Zebrafish aprt gene, the Yersinia pestis aprt gene, the Xenopus tropicalis aprt gene, the mold aprt gene, the Drosophila melanogaster aprt gene, the Saccharomyces cerevisiae aprt gene, the Schizosaccharomyces pombe aprt gene, the E. coli aprt gene, the Lactobacillus rhamnosus aprt gene and the Salmonella typhimurium aprt gene, to mention only a few. When the aprt gene of a hamster is desired, the hamster can be of any genus for example Mesocricetus, Phodopus, Cricetus, Cricetulus, Allocricetulus, Cansumys and Tscherskia. Any subspecies of a hamster can be used and may include Cricetulus griseus, Cricetulus sp., Cricetulus alticola, Cricetulus barabensis, Cricetulus kamensis, Cricetulus longicaudatus, Cricetulus migratorius and Cricetulus sokolovi. When the aprt gene of a mouse is desired, the mouse can be of any subspecies for example Mus musculus, Peromyscus leucopus, or Peromyscus maniculatus.

An “internal element”, as used in the context of the present invention, refers to a nucleotide stretch that prevents gene silencing by preventing DNA methylation of itself and the promoter region, which includes the enhancer and the minimal promoter.

In various embodiments, the one or more internal elements of the CpG island of the aprt gene can include one or more binding sites for the transcription factor Sp1. When the aprt gene is the hamster gene, the SP1 binding site of the CpG island of the aprt gene has the sequence: 5′-GCCCCGCCCCGTCCCGCCCC-3′ (SEQ ID NO: 1). When the aprt gene is the mouse aprt gene, the SP1 binding site of the CpG island of the aprt gene has the sequence 5′-CCCGCCC-3′ (SEQ ID NO: 2) or the sequence 5′-TCCGCCC-3′ (SEQ ID NO: 3). When the aprt gene is the hamster aprt gene, the internal element of the CpG island of the aprt gene has the sequence:

(SEQ ID NO: 4)
5′-TCCAGCAAATGCGTTACTTCCTGCCAAAAGCCAGCCTCCCCGCAACC
CACTCTCCCAGAGGCCCCGCCCCGTCCCGCCCCCTCCCGGCCTCTCCTCG
TGCTGGATCGCTCCCTAAGGA-3′.

When the aprt gene is the mouse aprt gene, the internal element of the CpG island of the aprt gene has the sequence:

(SEQ ID NO: 5)
5′-AGGATGGACATCGCACATCCCCTTTCCACCCATATATCTTTGAGGTA
GGGATGCTTGTGTTTAGGCAGCTCAAGAAATCTAACCCCTGACTCAGGCC
CCACACACACCTCGCAGAGGCCCCGCCTCTCAGCCTGTCCCGCCCCTCGT
GCTAGACCAACCCGCACCCAGAAGCCCCGCCCATCGAGGACGCTCCGCCC
TTGTTCCCCCCGGGATTGACGTG-3′.

In various embodiments, one or more internal elements of the CpG island of the aprt gene can be independently arranged in the nucleic acid molecule of the invention in the forward orientation (sense) or in the reverse orientation (antisense), relative to the sequence of the promoter. A plurality of internal elements for example two, three, four, five or more internal elements of the CpG island of the aprt gene and/or of the functional variant thereof can be independently arranged in the nucleic acid molecule of the invention.

In various embodiments, the nucleic acid molecule can further include an expressible nucleotide sequence coding for a polypeptide of interest. The expressible nucleotide sequence is operably linked to the promoter of the herpesvirus and the enhancer of the herpesvirus. An operable linkage is a linkage in which the regulatory DNA sequences according to the invention and the DNA sequences sought to be expressed are connected in such a way as to permit gene sequence expression. Therefore, the expressible nucleotide sequence can be transcribed from the functional promoter of the herpesvirus. The level of transcription is enhanced and/or stabilized over periods of about 10, about 20, about 30, about 40, about 50, about 60 or more cycles of expression by the internal element of the CpG island of an aprt gene and/or functional variant thereof.

In various embodiments, the expressible nucleotide sequence is a heterologous nucleotide sequence. The expressible nucleotide sequence can in some embodiments be arranged downstream from the promoter of the herpesvirus and the enhancer of the herpesvirus. The term “heterologous” when used in reference to a nucleotide sequence or nucleic acid molecule, means a nucleotide sequence not naturally occurring in the respective cell into which the nucleic acid molecule has been (or is being) introduced. A heterologous nucleic acid sequence thus originates from a source other than the respective cell and can occur naturally or non-naturally. A respective heterologous nucleic acid sequence may for example be integrated into the nucleic acid molecule of the present invention.

The functional promoter and the enhancer can be of the same herpesvirus or of different types of herpesviruses. The herpesvirus may be any herpesvirus. Non-limiting illustrative examples can include cytomegalovirus such as hCMV, a rhadinovirus, a roseolovirus, a simplexvirus, a varicellovirus, a mardivirus, an iltovirus, a betaherpesvirus, a gammaherpesvirus, an Epstein-Barr virus, a varicella zoster virus, murine gammaherpesvirus-68, Herpes Simian B virus, Herpes simplex-1 virus and Kaposi's sarcoma-associated herpes virus. In various embodiments, the cytomegalovirus is a human cytomegalovirus (hCMV).

The nucleic acid molecule of the herpesvirus contains a promoter region (which includes the functional enhancer and the minimal promoter) and can be of any length. In some embodiments, the promoter regions can be of a size of at least about 300 base pairs, a size of about 350 base pairs or more, about 400 base pairs or more, about 450 base pairs or more, about 500 base pairs or more, about 550 base pairs or more or about 600 base pairs or more.

In various embodiments, the functional promoter and the functional enhancer can be comprised in the sequence:

(SEQ ID NO: 6)
5′-TTGACATTGATTATTGACTAGTTATTAATAGTAATCAATTACGGGGT
CATTAGTTCATAGCCCATATATGGAGTTCCGCGTTACATAACTTACGGTA
AATGGCCCGCCTGGCTGACCGCCCAACGACCCCCGCCCATTGACGTCAAT
AATGACGTATGTTCCCATAGTAACGCCAATAGGGACTTTCCATTGACGTC
AATGGGTGGAGTATTTACGGTAAACTGCCCACTTGGCAGTACATCAAGTG
TATCATATGCCAAGTACGCCCCCTATTGACGTCAATGACGGTAAATGGCC
CGCCTGGCATTATGCCCAGTACATGACCTTATGGGACTTTCCTACTTGGC
AGTACATCTACGTATTAGTCATCGCTATTACTCGAGTGATGCGGTTTTGG
CAGTACATCAATGGGCGTGGATAGCGGTTTGACTCACGGGGATTTCCAAG
TCTCCACCCCATTGACGTCAATGGGAGTTTGTTTTGGCACCAAAATCAAC
GGGACTTTCCAAAATGTCGTAACAACTCCGCCCCATTGACGCAAATGGGC
GGTAGGCGTGTACGGTGGGAGGTCTATATAAGCAGAGCTC-3′.

In various embodiments, the functional promoter has the sequence:

(SEQ ID NO: 7)
5′-TGATGCGGTTTTGGCAGTACATCAATGGGCGTGGATAGCGGTTTGAC
TCACGGGGATTTCCAAGTCTCCACCCCATTGACGTCAATGGGAGTTTGTT
TTGGCACCAAAATCAACGGGACTTTCCAAAATGTCGTAACAACTCCGCCC
CATTGACGCAAATGGGCGGTAGGCGTGTACGGTGGGAGGTCTATATAAGC
AGAGCTC-3′.

In various embodiments, the functional enhancer has the sequence:

(SEQ ID NO: 8)
5′-TGACATTGATTATTGACTAGTTATTAATAGTAATCAATTACGGGGTC
ATTAGTTCATAGCCCATATATGGAGTTCCGCGTTACATAACTTACGGTAA
ATGGCCCGCCTGGCTGACCGCCCAACGACCCCCGCCCATTGACGTCAATA
ATGACGTATGTTCCCATAGTAACGCCAATAGGGACTTTCCATTGACGTCA
ATGGGTGGAGTATTTACGGTAAACTGCCCACTTGGCAGTACATCAAGTGT
ATCATATGCCAAGTACGCCCCCTATTGACGTCAATGACGGTAAATGGCCC
GCCTGGCATTATGCCCAGTACATGACCTTATGGGACTTTCCTACTTGGCA
GTACATCTACGTATTAGTCATCGCTATTA-3′.

Typically, the enhancer of the herpesvirus is arranged upstream of the promoter of the herpesvirus.

In various embodiments, at least one of the one or more internal elements of the CpG island of the aprt gene, or the functional variant thereof, is arranged upstream of the promoter of the herpesvirus or adjacently downstream of the promoter of the herpesvirus. When the one or more internal elements of the CpG island of the aprt gene, or the functional variant thereof is arranged upstream of the promoter of the herpesvirus, the said internal element(s) can be arranged (i) between the enhancer of the herpesvirus and the promoter of the herpesvirus or (ii) adjacently upstream of the enhancer of the herpesvirus.

In various embodiments, at least one of the one or more internal elements of the CpG island of the aprt gene, or the functional variant thereof, is arranged adjacent to the enhancer of the herpesvirus or adjacent to the promoter of the herpesvirus. “Adjacent” or “adjacently” as used herein in relation to nucleic acid sequence elements includes embodiments wherein the 3′ end of one sequence element directly connects to the 5′ of the other sequence element. In other embodiments, the respective sequence elements may be separated by less than 20 additional nucleotides that belong to neither the first nor the second sequence element, but rather define a linker-type sequence. If the two sequences are separated by such a linker-like sequence, they may still be in frame. In such embodiments, the linker-like additional nucleotide sequence may be 3, 6, 9, 12, 15, or 18 nucleotides long.

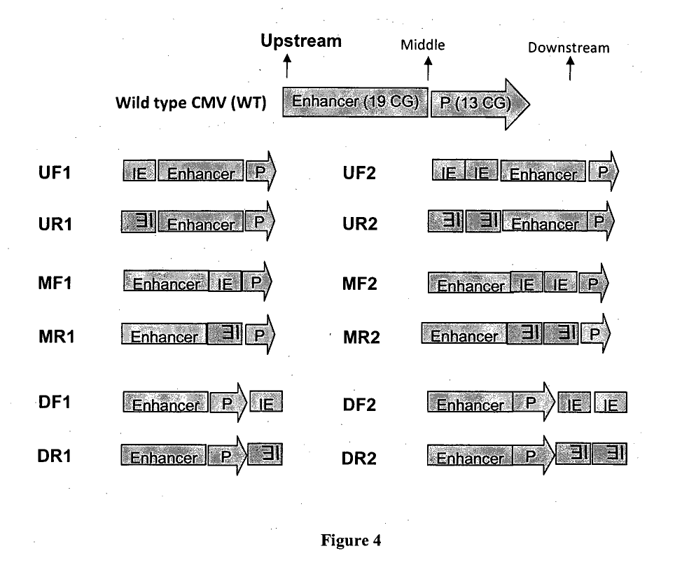

The one or more internal elements of the CpG island of the aprt gene can be independently arranged either in the forward (sense) or reverse (antisense) orientation in the nucleic acid molecule of the invention. In specific embodiments, the nucleic acid molecules of the invention can be illustrated in FIG. 4, wherein 1) one or more internal elements of the CpG island of the aprt gene or the functional variant thereof is arranged adjacently upstream of the enhancer of the herpesvirus in the forward or reverse orientations (see UF1, UF2, UR1 and UR2); 2) one or more internal elements of the CpG island of the aprt gene or the functional variant thereof is arranged between the enhancer and the promoter of the herpesvirus in the forward or reverse orientations (see MF1, MF2, MR1 and MR2); and 3) and one or more internal elements of the CpG island of the aprt gene or the functional variant thereof is arranged adjacently downstream of the promoter of the herpesvirus in the forward or reverse orientations (see DF1, DF2, DR1 and DR2).

As used herein, “nucleic acid” and “nucleic acid molecule” are used interchangeably and refer to any acid in any possible configuration, such as linearized single stranded, double stranded or a combination thereof. Nucleic acids may include, but are not limited to DNA molecules (e.g., cDNA or genomic DNA), RNA molecules (e.g., mRNA), analogues of the DNA or RNA generated using nucleotide analogues or using nucleic acid chemistry, cDNA synthetic DNA, a copolymer of DNA and RNA, oligonucleotides, and PNA (protein nucleic acids). DNA or RNA may be of genomic or synthetic origin and may be single or double stranded. A respective nucleic acid may furthermore contain non-natural nucleotide analogues and/or be linked to an affinity tag or a label. Also encompassed are nucleic acid analogues, such as peptide nucleic acids and the like.

As used herein, the general term “nucleotides” include nucleoside mono-, di-, and triphosphates. However, in the context of a nucleotide sequence, i.e. an oligo- or polynucleotide, the nucleotides are polymerized with a phosphate backbone, i.e. are monophosphates. The term “nucleotides” also includes modified nucleotides, such as, but not limited to, phosphorothioate nucleotides and deazapurine nucleotides, 2′-methoxy nucleotides and other nucleotide analogs.

In various embodiments, the nucleic acid molecule according to the invention is a DNA molecule. In some embodiments, the functional promoter of the herpesvirus is the only promoter comprised in the nucleic acid molecule.

In various embodiments, the nucleic acid molecule of the invention is comprised in a vector. The term “vector” relates to a single or double-stranded circular nucleic acid molecule that can be introduced, e.g. transfected, into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule according to the present invention can be inserted into a vector by cutting the vector with restriction enzymes and ligating the two pieces together. Therefore, the invention also provides a vector comprising a nucleic acid molecule of the invention. In some embodiments, the vector is an eukaryotic or a prokaryotic expression vector. In other embodiments, the vector is a plasmid. Commercially available plasmids such as pBR322, puC19, pBluescript and the like may be used. Typically, mammalian expression vectors can be used and are commercially available. Examples of mammalian expression vectors can include but are not limited to pCDM8 (Seed, B. Nature, 1987, 329: 840), pMT2PC (Kaufman et al, 1987, EMBO J. 6:187-195), pCI and pSI mammalian vectors.

In some embodiments, the nucleic acid molecule is comprised in a suitable host cell. The host cell comprising the nucleic acid molecule of the invention can be an eukaryotic or a prokaryotic cell. In this context, the (transformed) host cells can be cultured under conditions suitable for expression of the nucleic acid molecule of the invention. Host cells can be established, adapted and completely cultivated under serum free conditions, and optionally in media which are free of any protein/peptide of animal origin. Commercially available media such as RPMI-1640 (Sigma), Dulbecco's Modified Eagle's Medium (DMEM; Sigma), Minimal Essential Medium (MEM; Sigma), CHO-S-SFMII (Invitrogen), serum free-CHO Medium (Sigma), and protein-free CHO Medium (Sigma) are exemplary appropriate nutrient solutions. Any of the media may be supplemented as necessary with a variety of compounds, examples of which are hormones and/or other growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics, trace elements. Any other necessary supplements may also be included at appropriate concentrations that are known to those skilled in the art.

In some embodiments, the nucleic acid molecule is an isolated nucleic acid molecule. An “isolated” nucleic molecule of the present invention can refer to one that is separated from other nucleic acid molecules which are present in the natural source of the nucleic acid molecule. An isolated molecule, for example a DNA molecule, can be substantially, free of other cellular material, or culture medium when produced by recombinant techniques, or substantially free of chemical precursors or other chemicals when chemically synthesized. In other words, an isolated nucleic acid molecule can be free or substantially free of sequences (for example protein-encoding sequences) which flank the nucleic acid (i.e., sequences located at the 5′ and 3's ends of the nucleic acid) in the genomic DNA of a cell or organism from which the nucleic acid is derived.

Typically, the term “functional variant”, as used herein, refers to a sequence with at least 75%, at least 80%, at least 85%, at least 90%, at least 95%, at least 97%, or at least 99% sequence, or 75%, 80%, 85%, 90%, 91%, 92%, 93%, 94%, 95%, 96%, 97%, 98%, 99% identity to its respective original sequence, i.e. usually the corresponding aprt gene. By “sequence identity” is meant a property of sequences that measures their similarity or relationship. This term refers to the percentage of pair-wise identical residues obtained after a homology alignment of a nucleic acid sequence, of an aprt gene with a nucleic acid sequence, respectively, in question, wherein the percentage figure refers to the number of residues in the longer of the two sequences.

Also encompassed by the present invention are nucleic acid sequences substantially complementary to the above nucleic acid sequence. “Substantially complementary” as used herein refers to the fact that a given nucleic acid sequence is at least 90, for instance at least 95, and in some embodiments 100% complementary to another nucleic acid sequence. The term “complementary” or “complement” refers to two nucleotides that can form multiple favourable interactions with one another. Such favourable interactions include and may exclusively be Watson-Crick base pairing. A nucleotide sequence is the full complement of another nucleotide sequence if all of the nucleotides of the first sequence are complementary to all of the nucleotides of the second sequence.

The invention also relates to a method of producing a desired polypeptide. The method comprising: providing a nucleic acid molecule according to the present invention, wherein the nucleic acid molecule further comprises a nucleotide sequence coding for the desired polypeptide, the nucleotide sequence being operably linked to the promoter of the herpesvirus and the enhancer of the herpesvirus, and allowing expression of the desired polypeptide.

The term “polypeptide” as used herein is intended to cover naturally occurring proteins, as well as those which are recombinantly or synthetically produced. The term “polypeptide” is not limited to a specific length of the encoded product and, therefore, encompasses peptides, oligopeptides, and proteins. The term “polypeptide” also encompasses two or more polypeptides combined to form the encoded product. Polypeptides also include hybrid polypeptides which comprise a combination of partial or complete polypeptide sequences obtained from at least two different polypeptides wherein one or more may be heterologous to the cell in which the polypeptide is expressed. Polypeptides further include naturally occurring allelic and engineered variations of the above mentioned polypeptides and hybrid polypeptides.

In some embodiments, the nucleotide sequence encoding the desired polypeptide is a heterologous nucleotide sequence.

In some embodiments, the step of allowing expression of the desired polypeptide comprises introducing the nucleic acid molecule according to the present invention into a suitable host cell. In some embodiments, the nucleic acid molecule according to the present invention is comprised in a vector suitable for expression in the host cell.

The host cell may be any suitable cell, such as a eukaryotic cell. The eukaryotic cell can, for example be an animal cell, a plant cell (for e.g. monocots, dicots, algae), a fungus, a yeast cell, flagellum, microsporidia or protist. An animal cell can be derived from a mammal such as a primate, human, murine, bovine, rodent, human, insect, reptile, or a bird, to mention only a few. Examples of an animal cell such as a mammalian cell can include Chinese hamster ovary cells (for e.g. CHO-K1), COS cells (e.g., COS-1, COS-7), baby hamster kidney cells (BHK), human embryonic kidney (HEK) (e.g. HEK 293), Bowes melanoma cells, rat myeloma cells, mouse myeloma cells, antibody producing-hybridoma cells, human leukemia cells and the like. In some embodiments, the host cell is a neuron. In other embodiments, the host cell is a stem cell. A yeast cell can for example be S. cerevisiae, S. pombe, C. albicans, or Saccharomycetale cell.

In some embodiments, expression of the desired polypeptide is allowed in the host cell for over about 30 generations or more. Within the context of this embodiment, the level of expression of the desired polypeptide after about 30 generations or more of the host cell is enhanced when compared to a nucleic acid molecule with a heterologous nucleotide sequence coding for the polypeptide of interest that does not have an internal element of the CpG island of the aprt gene and/or a functional variant thereof.

In some embodiments, the desired polypeptide is expressed in a plurality of host cells, and wherein the number of host cells still expressing the desired polypeptide after about 30 generations or more of the host cell may be enhanced when compared to a plurality of host cells comprising a nucleic acid molecule with a heterologous nucleotide sequence coding for the polypeptide of interest that does not have an internal element of the CpG island of the aprt gene and/or a functional variant thereof.

In some embodiments, expression of the desired polypeptide is allowed over a period of about four weeks or more. Within the context of this embodiment, the level of expression of the desired polypeptide after the period of four weeks or more may be enhanced when compared to a nucleic acid molecule with a heterologous nucleotide sequence coding for the polypeptide of interest that does not have an internal element of the CpG island of the aprt gene and/or a functional variant thereof.

In some embodiments, the desired polypeptide is expressed in a plurality of host cells, and wherein the number of host cells still expressing the desired polypeptide after the period of four weeks or more is enhanced when compared to a plurality of host cells comprising a nucleic acid molecule with a heterologous nucleotide sequence coding for the polypeptide of interest that does not have an internal element of the CpG island of the aprt gene and/or a functional variant thereof.

In some embodiments, expression of the desired polypeptide is allowed in the host cell for over about 60 generations or more.

In some embodiments, expression of the desired polypeptide is allowed over a period of eight weeks or more. Within the context of this embodiment, the level of expression of the desired polypeptide after the period of eight weeks or more is enhanced when compared to a nucleic acid molecule that does not have an internal element of the CpG island of the aprt gene and/or a functional variant thereof.

In some embodiments, the desired polypeptide is expressed in a plurality of host cells, and wherein the number of host cells still expressing the desired polypeptide after the period of eight weeks or more is enhanced when compared to a plurality of host cells comprising a nucleic acid molecule with a heterologous nucleotide sequence coding for the polypeptide of interest that does not have an internal element of the CpG island of the aprt gene and/or a functional variant thereof.

The invention also provides a use of the nucleic acid molecule according to the present invention for enhancing the expression of a polypeptide of interest, wherein the nucleic acid molecule comprises a nucleotide sequence coding for the polypeptide of interest, the nucleotide sequence being operably linked to the promoter of the herpesvirus and the enhancer of the herpesvirus.

In some embodiments, polypeptide of interest is expressed in a suitable host cell. In some embodiments expression of the polypeptide of interest is allowed in the host cell over about 30 generations or more. In some embodiments, expression of the polypeptide of interest is allowed in the host cell over about 60 generations or more.

In some embodiments, the level of expression of the desired polypeptide after about 30 generations or more of the host cell is enhanced when compared to a nucleic acid molecule with a heterologous nucleotide sequence coding for the polypeptide of interest that does not have an internal element of the CpG island of the aprt gene and/or a functional variant thereof.

In some embodiments, the level of expression of the desired polypeptide after about 60 generations or more of the host cell is enhanced when compared to a nucleic acid molecule with a heterologous nucleotide sequence coding for the polypeptide of interest that does not have an internal element of the CpG island of the aprt gene and/or a functional variant thereof.

In some embodiments, the desired polypeptide is expressed in a plurality of host cells, and wherein the number of host cells still expressing the desired polypeptide after about 30 generations or more of the host cell is enhanced when compared to a plurality of host cells comprising a nucleic acid molecule with a heterologous nucleotide sequence coding for the polypeptide of interest that does not have an internal element of the CpG island of the aprt gene and/or a functional variant thereof.

In some embodiments, the desired polypeptide is expressed in a plurality of host cells, and wherein the number of host cells still expressing the desired polypeptide after about 60 generations or more of the host cell is enhanced when compared to a plurality of host cells comprising a nucleic acid molecule with a heterologous nucleotide sequence coding for the polypeptide of interest that does not have an internal element of the CpG island of the aprt gene and/or a functional variant thereof.

In some embodiments, the internal element of the CpG island of the aprt gene and/or the functional variant thereof prevents methylation of the functional promoter and/or the functional enhancer, thereby decreasing and/or preventing the reduction of the level of expression of the desired polypeptide.

The nucleic acid molecule of the present invention can for instance be used in recombinant protein production, for example, in mammalian cells, cell engineering, protein engineering or gene therapy.

The following exemplary data illustrates the usefulness and advantages of a nucleic acid molecule according to the present invention.

### EXAMPLES

### Generation of IE CMV Promoters

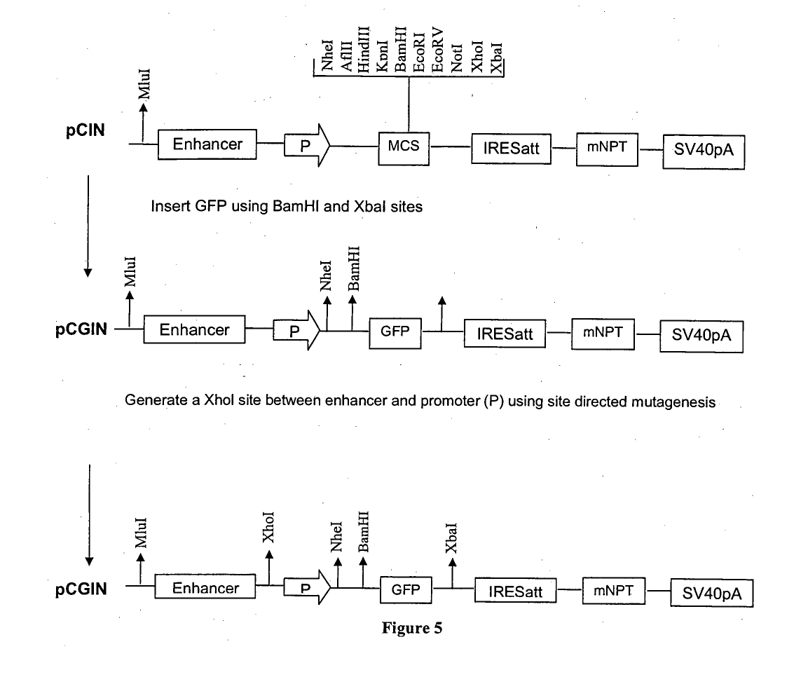

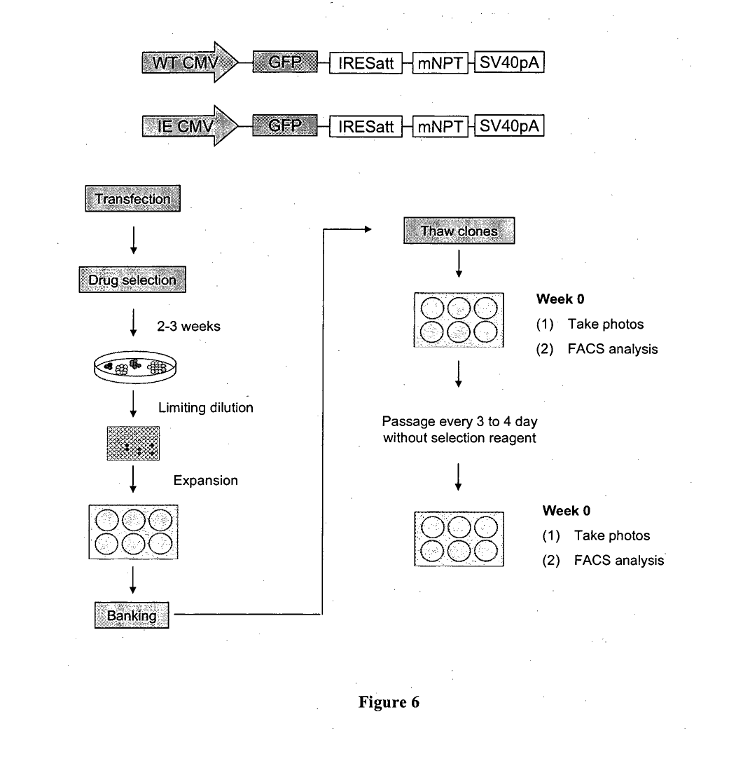

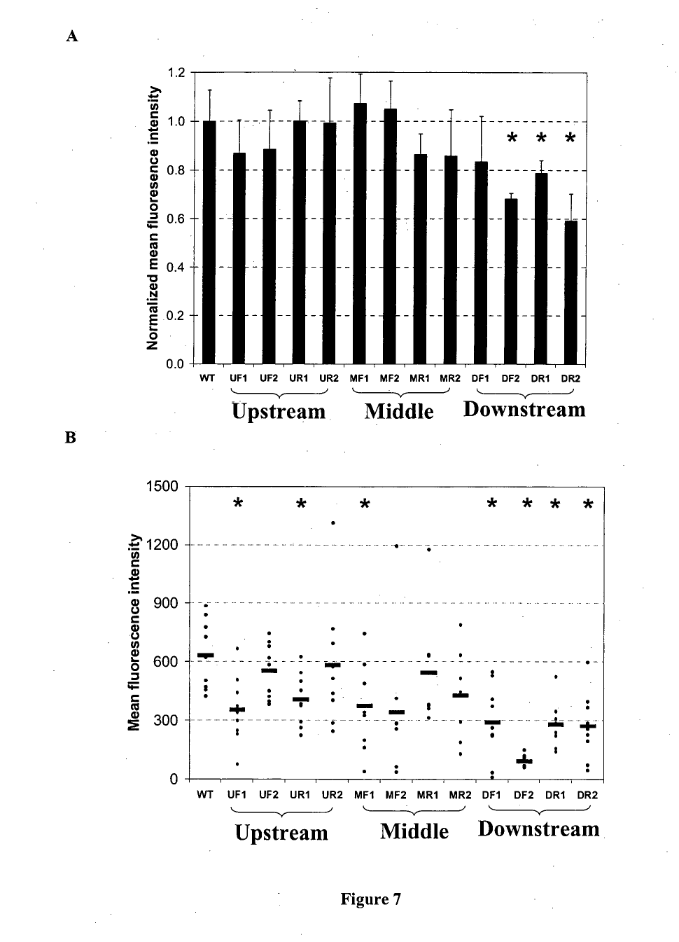

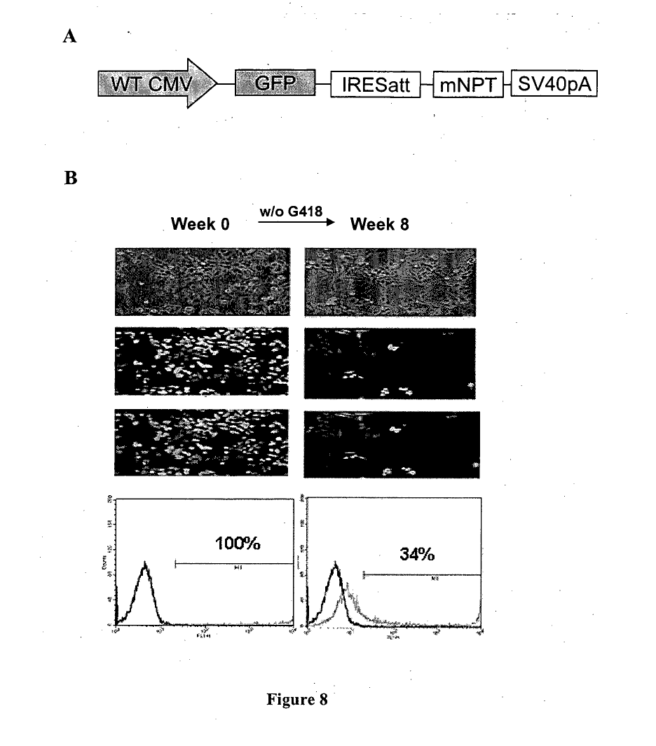

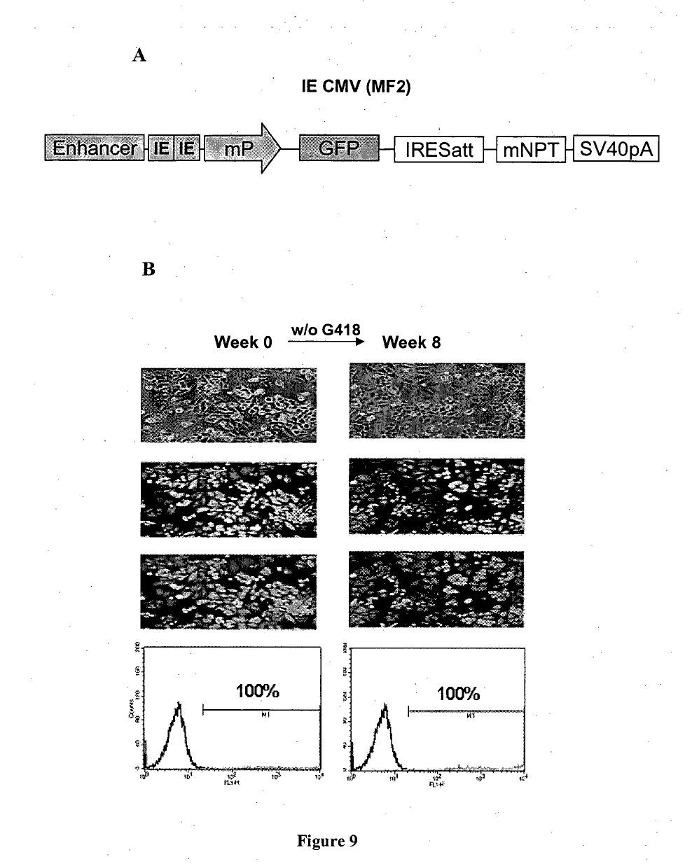

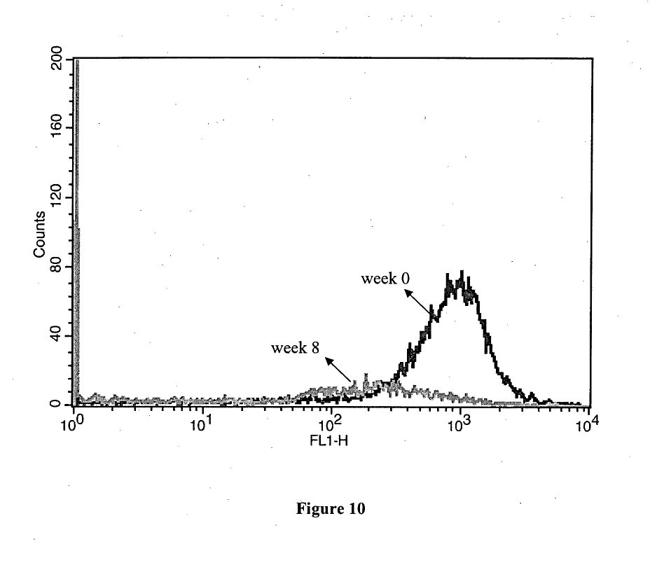

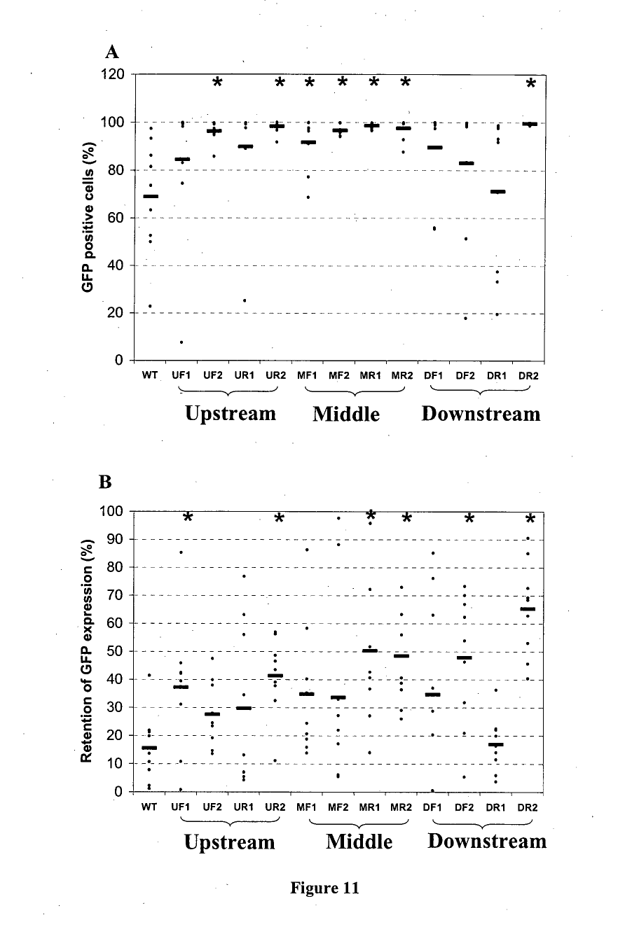

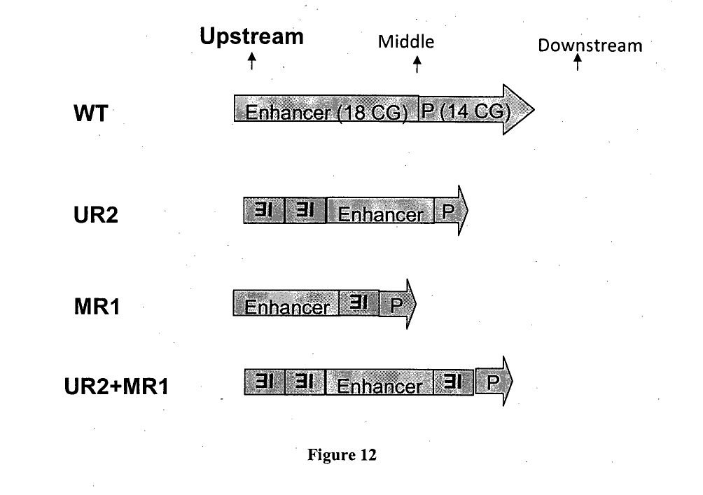

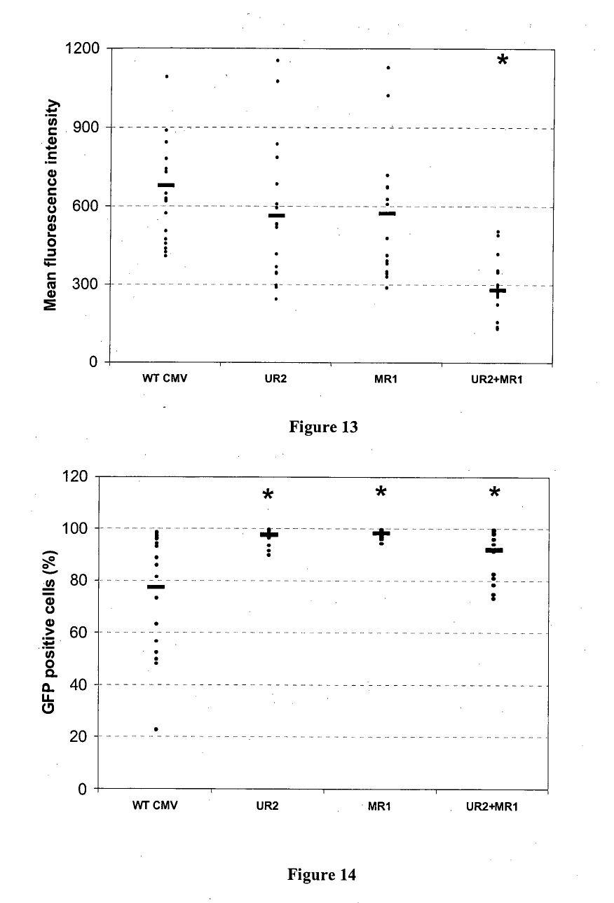

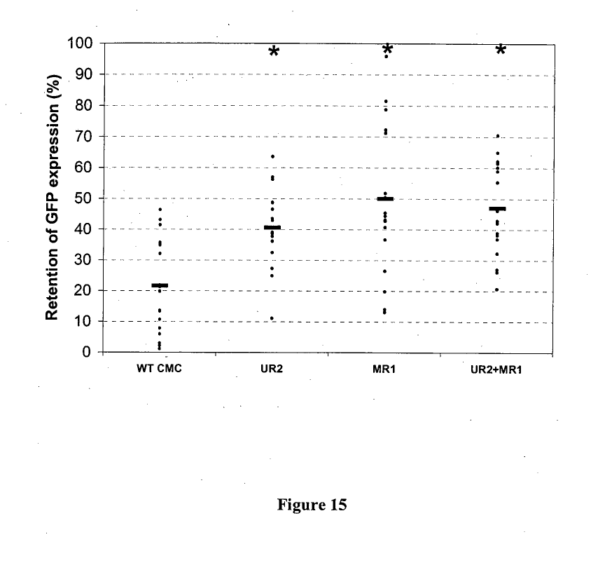

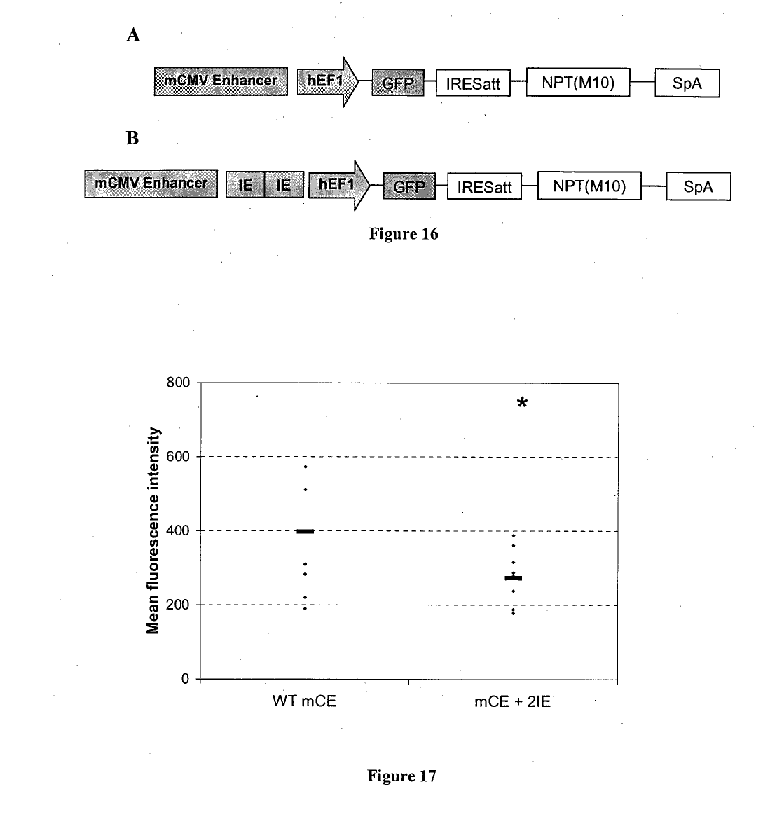

Generation of IE CMV and vectors for testing their ability to maintain gene expression during long term culture was summarized in FIGS. 4 and 5. The IE element was synthesized using PCR to anneal two primers,

### IE-Forward: 5′-TCCAGCAAATGCGTTACTTCCTGCCAAAAGCCAGCCTCCCCGCAACCCACTCTCC CAGAGGCCCCGCCCC-3′ (SEQ ID NO: 12) and

### IE-Reverse: 5′-TCCTTAGGGAGCGATCCAGCACGAGGAGAGGCCGGGAGGGGGCGGGACGGGGC GGGGCCTCTGGGAGA-3′ (SEQ ID NO: 13).

Two copies of IE were linked by overlapping PCR. The wild type CMV promoter in pCIN was cloned from pcDNA3.1(+) vector (Invitrogen, Carlsbad, Calif.). To generate IE CMV, a XhoI site was generated between CMV enhancer and minimal promoter (mP) using QuickChange site directed mutagenesis kit (Stratagene, La Jolla, Calif.). One copy or two copies of IE in either forward or reverse orientation were then inserted into upstream of CMV promoter using MluI site, between enhancer and mP using XhoI site, and downstream of CMV promoter using NheI site. All restriction enzymes and ligase used for vector construction were ordered from New England Biolabs (Ipswich, Mass.).

### FACS Analysis of GFP Expression in CHO Cells

The FACSCalibur™ system (Becton Dickinson, CA, USA) was used for estimating the green fluorescence levels of various clones. Green fluorescence at 525 nM was detected through FL1 set at a PMT voltage of 269 or 516, with a logarithmic gain. Ten thousand cells were analyzed for each sample. Data analysis was performed using Flowjo software.

### Analysis of GFP Gene Copy Number and mRNA Levels

Genomic DNA and RNA were isolated from cells using PureGene DNA purification kit (Gentra Systems, Minneapolis, Minn.) and RNAqueous-4PCR kit (Ambion, Austin, Tex.), respectively. First-strand cDNA was prepared from mRNA using the ImProm-II Reverse Transcription System (Promega, Madison, Wis.). For each reverse transcription reaction 100 ng total RNA were used. The relative GFP transgene copy numbers and mRNA levels were determined using real-time quantitative PCR (qRT-PCR). β-actin, a housekeeping gene, served as the internal control to normalize for possible variation in input quantity of DNA or RNA between samples. qRT-PCR was performed by using the ABI Prism 7000 sequence Detection System in conjunction with the iTag SYBR Green Supermix (Bio-Rad, Hercules, Calif.). The collected data were analyzed using the 2^{-ΔΔct} method (Livak and Schittgen, 2001, Methods 25 (4): 402-408).

### Example 1

### Generation of Single-Cell Clones

The vectors containing different modified CMV promoters with IE inserted are transfected into CHO K1 cells using electroporation in 6-well plate. At 48 hr post-transfection, G418, the selection reagent, is added for selection of cells with vectors integrated into genome. After 2 to 3 weeks selection, most survived cells will stably express GFP. Single-cell clones are then obtained using limiting dilution method and banked.

### Example 2

### Testing of Long Term Gene Expression

Single-cell clones obtained in step 1 are thawed and grown in 6-well plate. At week 0, photos are taken for different clones under the microscopes to estimate percentage of GFP positive cells. The percentage of GFP positive cells for each clone and expression level are also quantitatively determined using FAC. The cells are then passaged in the absence of G418 for 8 weeks. Photos are taken and FACS analysis is done again and compared results at week 0.

A modified hCMV promoter which is more resistant to gene silencing by using a core CpG island element (IE) of the aprt gene is described herein. The wild type hCMV promoter consists of an enhancer and promoter. Without wishing to be bound by any theory, it is found that insertions of a single IE element upstream of the enhancer or between the enhancer and promoter improve the ability of hCMV to maintain long term gene expression and do not compromise promoter strength for high level expression. It is also found that insertion of IE downstream of the minimal promoter also protects the hCMV from gene silencing but inhibits gene expression. Therefore and without wishing to be bound by theory, the protective effect of IE at each location is different and dependent on its orientation.

Another advantage of using the nucleic acid molecules of the present invention is the generation of cell lines with high stability. All clones generated using this modified CMV contained 100% GFP positive cells and maintained GFP expression over 50% of the original level after 8 weeks passaging, as compared to clones generated using the wild type CMV which contained many non-expressing cells and maintained expression less than 20%. Also, this modified IE CMV has comparable strength with the wild type CMV in expressing a gene in both transient and stable transfections. This anti-silencing protection provided by the IE element should be beneficial for generation of cell lines with high stability.

The listing or discussion of a previously published document in this specification should not necessarily be taken as an acknowledgement that the document is part of the state of the art or is common general knowledge. All documents listed are hereby incorporated herein by reference.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by a preferred embodiment, modification and variation of the invention herein disclosed may be resorted to by those skilled in the art, and that such modifications and variations are considered to be within the scope of this invention.

Other embodiments are within the following claims. In addition, where features or aspects of the invention are described in terms of Markush groups, the skilled artisan will recognize that the invention is also thereby described in terms of any individual member or subgroup of members of the Markush group.

1. A nucleic acid molecule comprising a functional promoter of a herpesvirus, a functional enhancer of a herpesvirus, and one or more internal elements of the CpG island of the aprt (adenine phosphoribosyl transferase) gene and/or a functional variant thereof.

2. The nucleic acid molecule of claim 1, wherein the aprt gene is one of the hamster aprt gene, the mouse aprt gene, the rat aprt gene, the human aprt gene, the bovine aprt gene, the Zebrafish aprt gene, the Yersinia pestis aprt gene, the Xenopus tropicalis aprt gene, the mold aprt gene, the Drosophila melanogaster aprt gene, the Saccharomyces cerevisiae aprt gene, the Schizosaccharomyces pombe aprt gene, the E. coli aprt gene, the Lactobacillus rhamnosus aprt gene and the Salmonella typhimurium aprt gene.

3. (canceled)

4. The nucleic acid molecule of claim 1, wherein at least one of the one or more internal elements of the CpG island of the aprt gene comprises one or more binding sites for the transcription factor Sp1.

5. The nucleic acid molecule of claim 4, wherein the aprt gene is a hamster aprt gene and the SP1 binding site of the CpG island of the aprt gene has the sequence: 5′-GCCCCGCCCCGTCCCGCCCC-3′(SEQ ID NO: 1).

6. The nucleic acid molecule of claim 4, wherein the aprt gene is a mouse aprt gene and the SP 1 binding site of the CpG island of the aprt gene has the sequence 5′-CCCGCCC-3′ (SEQ ID NO: 2) or the sequence 5′-TCCGCCC-3′ (SEQ ID NO: 3).

7. The nucleic acid molecule of claim 1, wherein the aprt gene is a hamster aprt gene and the internal element of the CpG island of the aprt gene has the sequence:
(SEQ ID NO: 4)
5′-TCCAGCAAATGCGTTACTTCCTGCCAAAAGCCAGCCTCCCCGCAACC
CACTCTCCCAGAGGCCCCGCCCCGTCCCGCCCCCTCCCGGCCTCTCCTCG
TGCTGGATCGCTCCCTAAGGA-3′.

8. The nucleic acid molecule of claim 1, wherein the aprt gene is a mouse aprt gene and the internal element of the CpG island of the aprt gene has the sequence:
(SEQ ID NO: 5)
5′-AGGATGGACATCGCACATCCCCTTTCCACCCATATATCTTTGAGGTA
GGGATGCTTGTGTTTAGGCAGCTCAAGAAATCTAACCCCTGACTCAGGCC
CCACACACACCTCGCAGAGGCCCCGCCTCTCAGCCTGTCCCGCCCCTCGT
GCTAGACCAACCCGCACCCAGAAGCCCCGCCCATCGAGGACGCTCCGCCC
TTGTTCCCCCCGGGATTGACGTG-3′.

9. The nucleic acid molecule of claim 1, wherein the one or more internal elements of the CpG island of the aprt gene are independently arranged in the nucleic acid molecule in the forward orientation or in the reverse orientation, relative to the sequence of the promoter.

10. The nucleic acid molecule of claim 1, comprising a plurality of the internal element of the CpG island of the aprt gene and/or of the functional variant thereof.

11. The nucleic acid molecule of claim 1, further comprising an expressible nucleotide sequence coding for a polypeptide of interest, the expressible nucleotide sequence being operably linked to the promoter of the herpesvirus and the enhancer of the herpesvirus.

12. The nucleic acid molecule of claim 11, wherein the expressible nucleotide sequence is a heterologous nucleotide sequence.

13-16. (canceled)

17. The nucleic acid molecule of claim 1, wherein the functional promoter and the functional enhancer are comprised in the sequence:
(SEQ ID NO: 6)
5′-TTGACATTGATTATTGACTAGTTATTAATAGTAATCAATTACGGGGT
CATTAGTTCATAGCCCATATATGGAGTTCCGCGTTACATAACTTACGGTA
AATGGCCCGCCTGGCTGACCGCCCAACGACCCCCGCCCATTGACGTCAAT
AATGACGTATGTTCCCATAGTAACGCCAATAGGGACTTTCCATTGACGTC
AATGGGTGGAGTATTTACGGTAAACTGCCCACTTGGCAGTACATCAAGTG
TATCATATGCCAAGTACGCCCCCTATTGACGTCAATGACGGTAAATGGCC
CGCCTGGCATTATGCCCAGTACATGACCTTATGGGACTTTCCTACTTGGC
AGTACATCTACGTATTAGTCATCGCTATTACTCGAGTGATGCGGTTTTGG
CAGTACATCAATGGGCGTGGATAGCGGTTTGACTCACGGGGATTTCCAAG
TCTCCACCCCATTGACGTCAATGGGAGTTTGTTTTGGCACCAAAATCAAC
GGGACTTTCCAAAATGTCGTAACAACTCCGCCCCATTGACGCAAATGGGC
GGTAGGCGTGTACGGTGGGAGGTCTATATAAGCAGAGCTC-3′.

18. The nucleic acid molecule of claim 1, wherein the functional promoter has the sequence:
(SEQ ID NO: 7)
5′-TGATGCGGTTTTGGCAGTACATCAATGGGCGTGGATAGCGGTTTGAC
TCACGGGGATTTCCAAGTCTCCACCCCATTGACGTCAATGGGAGTTTGTT
TTGGCACCAAAATCAACGGGACTTTCCAAAATGTCGTAACAACTCCGCCC
CATTGACGCAAATGGGCGGTAGGCGTGTACGGTGGGAGGTCTATATAAGC
AGAGCTC-3′.

19. The nucleic acid molecule of claim 1, wherein the functional enhancer has the sequence:
(SEQ ID NO: 8)
5′-TGACATTGATTATTGACTAGTTATTAATAGTAATCAATTACGGGGTC
ATTAGTTCATAGCCCATATATGGAGTTCCGCGTTACATAACTTACGGTAA
ATGGCCCGCCTGGCTGACCGCCCAACGACCCCCGCCCATTGACGTCAATA
ATGACGTATGTTCCCATAGTAACGCCAATAGGGACTTTCCATTGACGTCA
ATGGGTGGAGTATTTACGGTAAACTGCCCACTTGGCAGTACATCAAGTGT
ATCATATGCCAAGTACGCCCCCTATTGACGTCAATGACGGTAAATGGCCC
GCCTGGCATTATGCCCAGTACATGACCTTATGGGACTTTCCTACTTGGCA
GTACATCTACGTATTAGTCATCGCTATTA-3′.

20-21. (canceled)

22. The nucleic acid molecule of claim 1, wherein at least one of the one or more internal elements of the CpG island of the aprt gene, or the functional variant thereof, is arranged (i) between the enhancer of the herpesvirus and the promoter of the herpesvirus or (ii) adjacently upstream of the enhancer of the herpesvirus.

23. The nucleic acid molecule of claim 1, wherein at least one of the one or more internal elements of the CpG island of the aprt gene, or the functional variant thereof, is arranged adjacent to the enhancer of the herpesvirus or adjacent to the promoter of the herpesvirus.

24. (canceled)

25. The nucleic acid molecule of claim 1, wherein the functional promoter of the herpesvirus is the only promoter comprised in the nucleic acid molecule.

26. The nucleic acid molecule of claim 1, wherein the nucleic acid molecule is comprised in a vector.

27-28. (canceled)

29. The nucleic acid molecule of claim 1, wherein the nucleic acid molecule is comprised in a suitable host cell.

30-31. (canceled)

32. A method of producing a desired polypeptide, the method comprising:
providing a nucleic acid molecule according to claim 1, wherein the nucleic acid molecule further comprises a nucleotide sequence coding for a desired polypeptide, the nucleotide sequence being operably linked to the promoter of the herpesvirus and the enhancer of the herpesvirus, and
allowing expression of the desired polypeptide.

33. The method of claim 32, wherein the nucleotide sequence coding for the desired polypeptide is a heterologous nucleotide sequence.

34. The method of claim 32, wherein allowing expression of the desired polypeptide comprises introducing the nucleic acid molecule into a suitable host cell.

35. The method of claim 34, wherein the nucleic acid molecule is comprised in a vector suitable for expression in the host cell.

36. The method of claim 34, wherein expression of the desired polypeptide is allowed in the host cell over about 30 generations or more.

37. The method of claim 34, wherein the level of expression of the desired polypeptide after about 30 generations or more of the host cell is enhanced when compared to a nucleic acid molecule with a heterologous nucleotide sequence coding for the polypeptide of interest that does not have an internal element of the CpG island of the aprt gene and/or a functional variant thereof.

38-39. (canceled)

40. The method of claim 34, wherein the level of expression of the desired polypeptide after the period of four weeks or more is enhanced when compared to a nucleic acid molecule with a heterologous nucleotide sequence coding for the polypeptide of interest that does not have an internal element of the CpG island of the aprt gene and/or a functional variant thereof.

41-45. (canceled)

46. A vector comprising the nucleic acid molecule according to claim 1.

47-58. (canceled)
